(19) United States
(12) Patent Application Publication
(10) Pub. No.: US 2008/0046047 A1
(43) Pub. Date: Feb. 21, 2008

(54) Hot and cold therapy device

(76) Inventor: Daniel Jacobs, New York, NY (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/507,392

(22) Filed: Aug. 21, 2006

(51) Int. Cl. A61F 7/00

(52) U.S. Cl. 607/108; 607/112

(57) ABSTRACT

A therapy device incorporating a thermoelectric (Peltier) cell to enable therapeutic temperatures to be applied to a body. As current passes through the cells, heat is transferred from one surface of the cell to the other. Opposite directional current changes the heat transfer direction. A heat sink may be in thermal communication with the thermoelectric cell to permit low temperatures to be achieved for a significant period of time without overheating of the thermoelectric cell. A fan may be positioned adjacent the heat sink to keep the heat sinks cool. In some arrangements, pouches containing a fluid are used as an intermediary between the thermoelectric cell and the body of the user. One arrangement includes multiple heating/cooling units installed into a vest for the targeted treatment of back problems.

### BACKGROUND OF THE INVENTION

1. Field of the Invention

The present invention relates generally to musculoskeletal therapy devices. More particularly, the present invention relates to a hot and cold therapy device that is relatively compact and offers good performance with relatively low power requirements.

2. Description of the Related Art

Injuries to the musculoskeletal system of an animal or human can result from many activities or occurrences. For example, injuries due to participation in sporting activities are quite common. Sporting injuries often occur to the back, the knees, the shoulders, the wrist, and the forearm, among other areas of the human body, or similar areas of non-human animal bodies. Extensive rehabilitation therapy sessions are routinely a part of the injury recovery process from many injuries, such as severe muscle strains, for example.

Most rehabilitation processes involve the application of heat to the injured area, followed by the application of cold to the injured area. The heat therapy and the cold therapy are normally separated by stretching and strengthening exercises. Once the specific therapy session has been devised by a healthcare professional, the necessary therapy steps could be completed conveniently at home. However, often the necessary supplies for the heat therapy and the cold therapy are available only at the healthcare professional's facilities. This typically necessitates travel on the part of the patient, and possibly long lines to gain access to the proper equipment. Thus, a device to provide both heat and cold therapy would be beneficial in permitting the rehabilitation to be completed away from the healthcare professional's facility, at a location convenient for the patient.

Some devices have been created to provide both heat and cold to the body of a user of the device. Some of these devices are intended to provide relief from excessively hot or cold external environments and, as a result, may not be capable of achieving the desired high and low temperatures, at least in a relatively portable configuration. Other devices are configured for therapeutic use, but generally suffer from other disadvantages such as not being capable of maintaining desired temperatures (hot or cold) for a sufficient period of time, or having excessive power demands.

### SUMMARY OF THE INVENTION

Preferred embodiments of the present invention provides a therapy device that is capable of achieving and maintaining desired hot and cold temperatures for a sufficient time to accomplish typical therapy goals. Furthermore, the preferred embodiments do not require excessive amounts of power to achieve and maintain such temperatures. The presently preferred embodiments are also relatively compact so as to be sufficiently portable. The preferred embodiments are also reasonably inexpensive so as to be realistic for personal use.

A preferred embodiment is a variable temperature therapy system for permitting ambulatory use on an animal. The device includes a support adapted to surround a portion of the body of a user of the device. The support is securable to the body of the user. A plurality of therapy units are coupled to the support and are located on the support so as to be positioned proximate target treatment areas of the body of the user when the device is secured to the user. Each temperature therapy unit includes a resilient pouch containing a non-circulating volume of a liquid. The resilient pouch is secured relative to the support. A thermoelectric heat pump is in thermal communication with the resilient pouch. A heat sink is coupled to a surface of the thermoelectric heat pump and a fan is configured to circulate air over the heat sink. The plurality of thermoelectric heat pumps is electrically connected with one another by an electric circuit, which is connectable to a power supply.

A preferred embodiment is a self-contained variable temperature therapy device for use on an animal including a support adapted to surround a portion of the body of a user of the device. The support is securable to the body of the user. A resilient pouch contains a volume of a liquid and is secured relative to the support so as to be proximate the body of the user when the support is secured to the user. A thermoelectric heat pump is connectable to a source of power. A heat sink has a first thermal resistance. A thermal compound layer is interposed between the heat sink and a surface of the thermoelectric heat pump. The thermal compound has a second thermal resistance that is less than or equal to the first thermal resistance. A cooling device is configured to withdraw heat from the heat sink.

A preferred embodiment is a variable temperature therapy device including a support adapted to surround a portion of the body of a user of the device. The support is securable to the body of the user and has a therapy surface configured to be adjacent a skin surface of the user when the device is in use. A resilient pouch contains a non-circulating volume of a liquid, the resilient pouch secured relative to the support. A thermoelectric heat pump is in thermal communication with the resilient pouch on an opposite side of the resilient pouch from the body of the user when the device is in use. The thermoelectric heat pump is connectable to a source of power. A heat sink is coupled to a surface of the thermoelectric heat pump. A fan is configured to circulate air over the heat sink. The device is configured such that the therapy surface is capable of maintaining a temperature at or below about 0 degrees Celsius for at least 20 minutes in a first setting and is capable of maintaining a temperature at or above about 46 degrees Celsius for at least 20 minutes in a second setting.

### BRIEF DESCRIPTION OF THE DRAWINGS

These and other features, aspects and advantages of the present invention are described below with reference to drawings of certain preferred embodiments, which are intended to illustrate, but not to limit, the present invention. The drawings contain nine figures.

FIG. 1 is an elevation view of a therapy device having certain features, aspects and advantages of the present invention.

FIG. 2 is a schematic view of a thermoelectric device and, specifically, a Peltier cell.

FIG. 3 is a graph of cold side temperature of the Peltier cell versus operating current.

FIG. 4 is a graph of heat pumped at the cold side versus operating current.

FIG. 5 is a rear view of a therapy device in the form of a vest.

FIG. 6 is a diagram of a preferred electrical circuit of the therapy device.

FIG. 7 is a diagram of a modified electrical circuit of the therapy device.

FIG. 8 is a graph of the temperature of the device over time.

FIG. 9 is a graph of the temperature of a user's back over time determined in a trial of the vest therapy device of FIG. 5.

### DETAILED DESCRIPTION OF THE PREFERRED EMBODIMENT

An objective of a preferred embodiment of the therapy device is to simplify the rehabilitation process. Preferably, the preferred embodiments permit an injured individual (or animal) to complete the necessary steps of therapy without being forced to visit a trainer or doctor's office. As many injuries are specific to the back, one preferred embodiment is tailored to treatment of that area. However, the core technology is adaptable to an injury to any part of the body. Preferably, the preferred embodiments simulate the traditional therapies in both temperature and sensation.

Typical Rehabilitation

As discussed above, even among varied injuries, the typical therapy protocol breaks down into a simple four-step process: heat, stretch, strengthen and ice. Each of these processes requires a certain piece of equipment to complete. All of the necessary tools can typically be found in a trainer's office. For the first step of the process, heating, a trainer will typically make use of a piece of equipment called a hydrocollator.

A hydrocollator is a large metal vat. It plugs into the wall and runs electricity through large metal coils inside the vat. Full to the brim with water, the vat is heated to about 80° C. via the coils. Resting in the water are heat pads. The heat pads absorb and retain heat from the hydrocollator water and are then placed on the injured area. The heating session usually lasts for around fifteen minutes. One pitfall of such heat pads is that they come out of the hydrocollator hot, but cool down by the end of the heating session.

The second stage of the therapy process is a long stretching. The stretching is followed by a regimen of rehabilitation or strengthening exercises. One must stretch before doing rehabilitation exercises to loosen up the muscles and prevent further strain. The purpose of these exercises is to rebuild the injured muscle, so it is important that they are warm and loose (from heat and stretching) before they are worked.

The final step in muscle rehabilitation therapy is an icing session. The icing session lasts roughly twenty minutes. A trainer may make use of an icebox with plastic bags or a chiller device similar to the hydrocollator; only the water and pads are maintained at very cold temperatures. The session can also be carried out with something as simple as a plastic bag full of ice from a refrigerator-freezer. Similar to the heat pads, these options do not typically maintain their target temperature of about 0° C. throughout the therapy session.

Preferred embodiments replace the hydrocollator pads and the bags of ice with an all-in-one therapy device, such as a vest, for example. To accomplish this, the preferred embodiments simulate the environments of the above-described treatments by producing the same temperature ranges as those traditional treatments. To match sensation of the heating session, the target temperature is roughly 46° C. To mimic ice, the target temperature is 0° C. Another goal was to avoid the pitfalls of the traditional therapies: maintain target temperature levels for the duration of the therapy session.

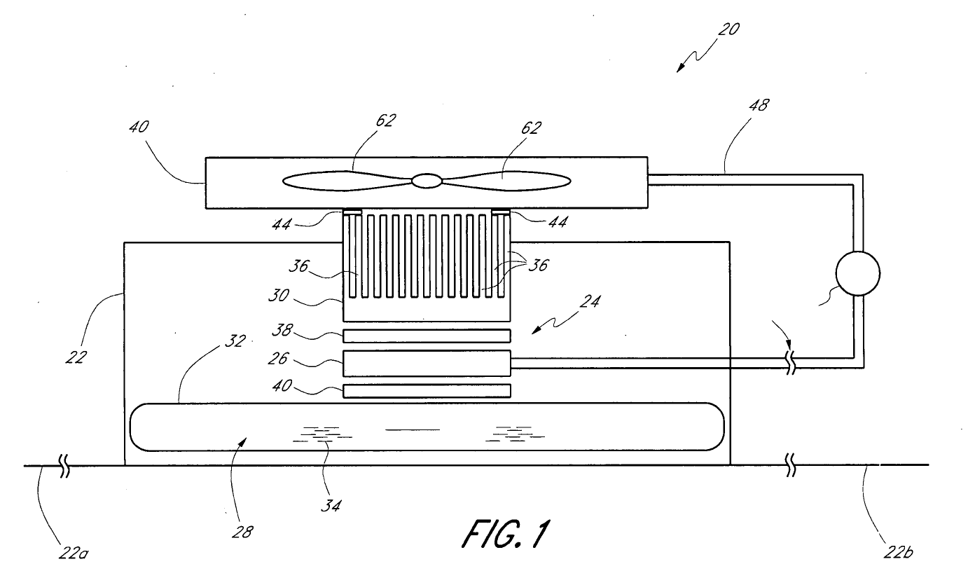

The Therapy Device of FIG. 1

FIG. 1 illustrates a preferred embodiment of the therapy device, which is referred to generally by the reference number 20. The therapy device preferably includes a support 22 and a therapy unit, or core unit 24. The therapy unit 24 is secured to the support 22, which is configured to permit the device 20 to be secured to a portion of the body of the user. In one arrangement, as discussed above, the support 22 may be a vest. However, in other arrangements, the support 22 may take on other suitable shapes to correspond to the shape of the target portion of the user's body.

In one arrangement, the support 22 is a wrap that may be secured around a portion of the body, such as a torso or limb. As illustrated, at least a portion of the therapy unit 24 is contained within a space of the support 22 between a first layer and a second layer of the support. The wrap may have end portions 22a, 22b that are capable of being secured to one another, such as by an interlocking hook and loop fastener, for example. Other suitable arrangements of the wrap will be apparent to those of skill in the art.

The support 22 may be made of any suitable material. For example, the support 22 may be constructed from a polymeric material, such as nylon. In other arrangements, such as a wrap configuration, the support 22 may be constructed of a neoprene material, similar to that commonly used for typical wraps or supports (e.g., ankle, knee, elbow supports).

The illustrated therapy unit 24 includes a thermoelectric device 26, a fluid reservoir 28 and a heat sink 30. As described above, preferably, the support 22 surrounds at least the thermoelectric device 26, fluid reservoir 28 and a portion of the heat sink 30 such that the therapy unit 24 is secured to and partially contained within the support 22. However, other suitable arrangements may be used to secure the therapy unit 24 to the support 22.

The thermoelectric device 26 is configured to generate heat or cold in response to electrical stimulation and, thus, is often referred to as a thermoelectric heat pump. More specifically, the thermoelectric device 26 preferably is a Peltier cell, which is configured to produce a temperature differential between its upper surface and lower surface (as illustrated in FIG. 1) in response to an electrical current being applied to the cell. Altering the direction of current alters the direction of flow of heat energy through the cell. Accordingly, the current direction may be controlled to control whether the lower surface of the cell is hot or cold.

The fluid reservoir 28 is faces the lower surface of the thermoelectric device 26 and advantageously acts as an energy transfer medium to transfer heat energy between the thermoelectric device 26 and the user of the therapy device 20. The illustrated fluid reservoir 28 includes a flexible or resilient pouch 32 containing a fixed volume of a heat transfer fluid 34. That is, preferably, the heat transfer fluid 34 is of a non-circulating arrangement that is directly heated or cooled by the thermoelectric device 26 at the therapy site. Other devices that employ a circulating fluid system, with a remote heating (or cooling) device and a fluid pump, typically are too complex and expensive to be affordable by the average consumer. Furthermore, such systems typically have too high of power requirements to be reasonably portable.

The fluid reservoir pouch 32 may be constructed from any suitable material, such as a thin polymer material, for example. Preferably, the pouch 32 is flexible or resilient such that it is relatively conformable to the targeted area of the body of the user. Such an arrangement maximizes heat transfer between the therapy device 20 and the user. The volume of fluid 34 contained within the reservoir pouch 32 may be altered depending upon the size of the target therapy site, the power of the thermoelectric device 26, or both. Thus, the thermoelectric device 26 heats or cools the volume of fluid 34, which in turn heats or cools the targeted area of the body of the user. In addition, the fluid reservoir 28 permits the heat energy generated by the thermoelectric device 26 to be applied to an area greater than the area of the thermoelectric device 26. As illustrated, a portion of the support 22 is positioned between the fluid reservoir 28 and the targeted area of the body of the user. However, in other arrangements, the support 22 may have an opening(s) (not shown) that permit direct contact between the fluid reservoir 28 and the user.

The heat sink 30 faces the upper surface of the thermoelectric device 26 and is configured to transfer heat energy away from the thermoelectric device 26. When electrical current is applied to the thermoelectric device 26, it tends to generate more heat than can be dispersed on its own, especially when being used to cool the fluid reservoir 28. Without a mechanism to increase the rate of heat dispersion, the entire thermoelectric device 26 would heat up, including the lower surface. Accordingly, the heat sink 30 is configured to remove excess heat from the thermoelectric device 26.

Preferably, the heat sink 30 is constructed from a material having a relatively low thermal resistance and, thus, permits the rapid movement of heat energy through the material. The heat sink 30 also has a relatively large surface area to volume ratio in comparison to the thermoelectric device 26. The illustrated heat sink 30 includes a plurality of fins 36 extending in an upward direction, which increases the overall surface area of the heat sink 30 to permit heat energy to be efficiently transferred to the surrounding atmosphere. However, other suitable arrangements to increase the surface area, or cooling power, of the heat sink 30 may also be employed.

Advantageously, the illustrated therapy unit 24 includes a first thermal compound layer 38 between the thermoelectric device 26 and the heat sink 30. Preferably, the therapy unit 24 also includes a second thermal compound layer 40 between the thermoelectric device 26 and the fluid reservoir 28. Desirably, each of the thermal compound layers 38, 40 directly contacts the components within which they are interposed. The thermal compound layers 38, 40 inhibit air gaps from existing between the components of the therapy unit 24 to increase the efficiency of the unit 24 and decrease the power required to achieve the desired therapy temperatures.

The thermal compound layers 38 may be constructed from any suitable material that is a good conductor of heat energy, as is described in greater detail below with respect to a vest embodiment of the therapy device. Desirably, however, the thermal resistance of the first thermal compound layer 38 is less than or equal to the thermal resistance of the heat sink 30 such that the rate of heat transfer from the thermoelectric device 26 to the surrounding atmosphere is determined by the heat sink 30 performance and not the first thermal compound layer 38. Similarly, it is desirable that the thermal resistance of the second thermal compound layer 40 is also less than or equal to the thermal resistance of the heat sink 30.

The therapy device 20 may also include a fan 42, or other air circulation device, configured to move air over the heat sink 30. Thus, the fan 42 increases the cooling performance of the heat sink 30. The fan 42 may be separated from the heat sink 30 by a spacer 44, such as pieces of a foam material, for example, to ensure that contact between moving parts of the fan 42 and the heat sink 30 does not occur. As illustrated, at least a portion of the fan 42 preferably is external of the support 22 such that direct access to atmospheric air is permitted. However, if desired, a portion of the fan 42 may be within the support 22 to assist in securing the fan 42 to the remaining components of the therapy unit 24.

The components of the therapy unit 24 may be coupled to one another and the support 22 by any suitable arrangement. For example, mechanical fasteners, adhesives or other suitable mechanisms, or any combination thereof, may be employed. Preferably, the components of the therapy unit 24 are secured to one another such that the therapy device 20 as a whole is relatively robust such that it is portable without being overly susceptible to damage.

A power source 46 provides power to the thermoelectric device 26 and the fan 40 through appropriate electrical connections 48. Although illustrated as a single component, the power source 46 may comprise multiple power sources or units. For example, each of the thermoelectric device 26 and the fan may be powered by separate power sources. In addition, a single power source, such as power source 46, may power multiple therapy units 24, including multiple thermoelectric devices 26 and fans 40. The power source 46 may be self-contained and portable (e.g., a battery) or may be stationary (e.g., a standard wall electrical outlet). Thus, the power source 46 is not necessarily an integral component of the therapy device 20, but may be a fitting, such as an electrical plug, configured to permit connection to an external power source. As discussed above, the therapy device 20 includes a switch 50, or other suitable structure, to permit the direction of current applied to the thermoelectric device 26 to be reversed, as will be appreciated by one of skill in the art.

The Peltier Cell

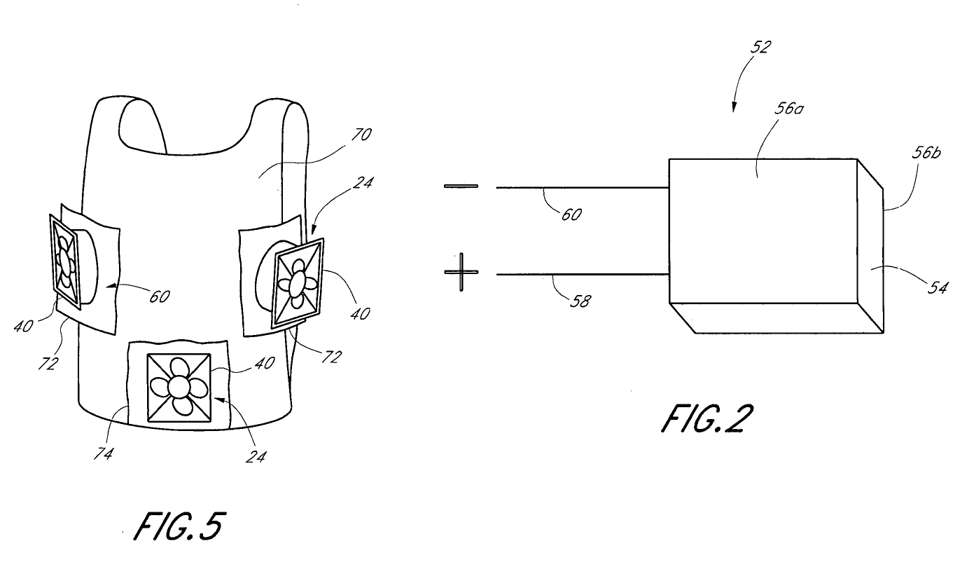

A schematic diagram of a preferred thermoelectric device 26, a Peltier cell 52, is shown in FIG. 2. The cell consists of semi-conductor material 54 sandwiched between two ceramic plates 56a, 56b. A positive lead 58 and a negative lead 60 extend from the Peltier cell 52.

These circuit elements serve two functions. By pumping heat into one side of these cells 52 and removing the same heat from the other side, one can generate a current through the cell 52. By the same token, Peltier cells 52 transfer heat from one plate 56a to the other plate 56b when a current is run through them. The latter is the means by which use is made of these cells 52 in the present therapy devices. As heat is removed from one side or plate 56a (or 56b) of the cell 52, it becomes cold. When the current is reversed, the heat is transferred in the opposite direction. This means the cells 52, when supplied with a regulated current, are capable of autonomous heating and cooling. By manipulating the current levels through these devices, the temperatures levels may be varied to reach the ranges desired for therapy.

Reaching high temperatures with a commercially available Peltier cell 52 with each plate 56a, 56b having an area of about 2.25 square inches is not a difficult task. Running current through almost any circuit element naturally causes that element to heat up. The real test for the Peltier cells 52 was to determine if they were capable of achieving temperatures cold enough to mimic ice. Use of software provided by the Peltier vendor illustrated that the standard Peltier cells 52 were capable of attaining the target cold temperature, as illustrated in FIG. 3, which is a graph of Cold Side Temperature (° C.) vs. Operating Current (A). As apparent from the graph of FIG. 3, the target temperature of 0° C. is well within the range of capability of the cell. Correlating the target temperature on the graph, it appears the Peltier cell requires a current of around 2.6 A.

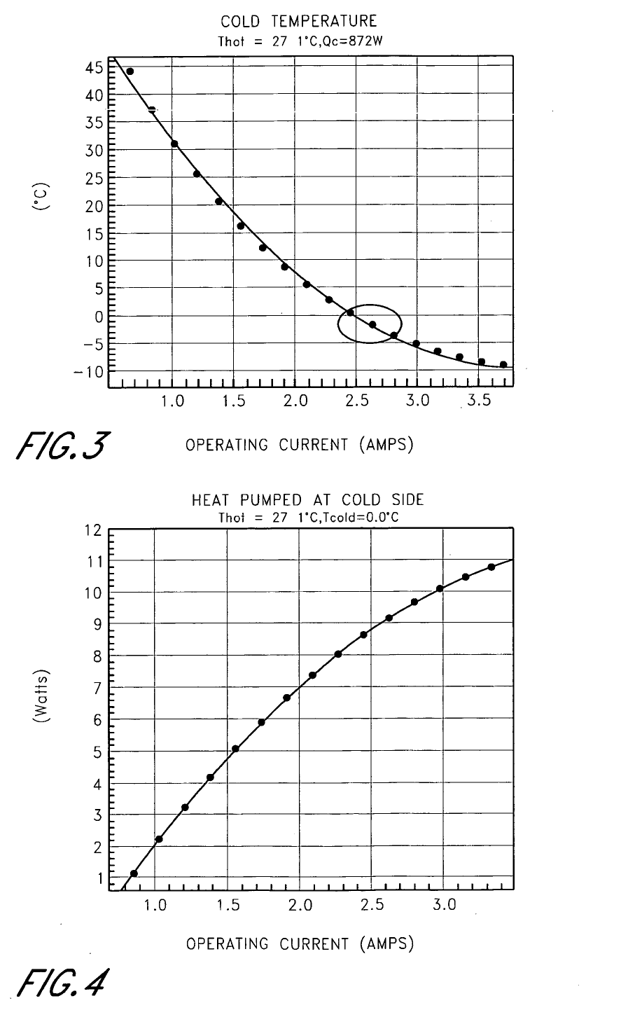

As discussed above, the Peltier cell 52 produce a cold sensation by transferring heat away from one surface. As illustrated in FIG. 4, it is apparent that when 2.6 A are being run through the Peltier cell 52, 9 W of heat is transferred away from the surface of the cold plate 56a or 56b. Without a way to remove that heat, it will remain on the cold plate 56a or 56b and heat up the entire cell 52. In preliminary lab testing, it was discovered that the entire cell 52 heated up within a period of time on the order of 10 seconds. Without some means of removing the transferred heat, the Peltier cells 52 would be rendered practically useless for performing a cooling function over a period of time sufficient to provide therapeutic results.

The Therapy Unit

To handle the excess heat being transferred across the cell 52, a tool for heat absorption was needed. Initial testing showed that a heat sink, such as the heat sink 30 of FIG. 1, served as the best method of heat removal. According to the Peltier cell manufacturer's software, combined with lab testing, a specific size heat sink was determined to match the heat transferred through the therapy unit 24. In one preferred arrangement, the heat sink 30 is a 16-fin, aluminum heat sink, measuring approximately 13×13×2 cm. The thermal resistance of the heat sink 30 preferably is less than or equal to 0.07 C-in²/W and the heat sink 30 preferably is large enough to allow an airflow of at least 102 cubic feet per minute (CFM). However, the properties of the heat sink 30 may be varied depending on the characteristics of the Peltier cell 52 and/or the desired use of the therapy device 20.

In initial testing, after a certain period of running time, the heat sink 30 began to reach its capacity for absorption. At this point, the heat sink 30 temperature began to rise significantly above room temperature of about 26° C. A way to keep the heat sink cool over long periods of time, to allow the cold side of the Peltier cell 52 to continue to lose heat was needed. For this reason, a fan 40 preferably is installed on the top of the therapy unit 24, as discussed above in connection with FIG. 1. The purpose of the fan 40 is to pass air between the fins 36 of the heat sink 30, keeping it cool via convection. As the heat sink 30 preferably is capable of airflow of at least 102 CFM, the fan 40 preferably is capable of pumping at least a similar amount of air. During testing, it was found that even after 30 minutes of continuous running time, the fins 36 of the heat sink 30 did not rise above room temperature with such an arrangement.

To secure the fan 40, heat sink 30 and Peltier cell 52 in one piece, two holes (not shown) may be drilled in the bottom of the heat sink 30, marginally wider than the Peltier cell 52. On the opposite side, aluminum strips (not shown) may be cut and shaped to line up with the holes in the heat sink 30, as well as standard mounting holes disposed each of the bottom corners of the fan 40 (not shown). Using fasteners, such as a flat head brass screws, the Peltier cell 52 may be held in place, screwing up through the bottom of the heat sink 30 to the aluminum strips on top. Such a system, or other suitable arrangements, may be used to hold the fan 40 on top of the therapy unit 24. To keep the blades 62 (FIG. 1) of the fan 40 from making contact with the fins 36, ½ inch thick pieces of foam, or other suitable materials, may be used as a spacer 44 (FIG. 1) to keep the fan 40 and heat sink 30 slightly apart, but still snuggly together. These units of a Peltier cell 52, a heat sink 30 and a fan 40 may be referred to herein as the therapy “core technology.”

As discussed above, it is preferable that the heat transfer between the surface of the hot plate 56a or 56b of the Peltier cell 52 and the heat sink 30 is maximized. Because the heat sink 30 generally is the limiting factor in removing heat from the system, it is desirable that the thermal resistance at the contact area between the heat sink 30 and the Peltier cell 52 is at most the thermal resistance of the heat sink such that air or anything else in between the cell 52 and the heat sink 52 does not interfere with heat flow. For this reason, preferably a layer 38 (FIG. 1) of thermal compound is spread onto the Peltier cell 52 before attaching it to the heat sink 30. A preferred thermal compound includes silver and, in one arrangement, is composed of 99% silver has a thermal resistance of about 0.07 C-in2/W, or essentially the same as the heat sink 30. The thermal compound may have a thermal resistance that is less than the heat sink 30 as well. Since the preferred thermal compound material is more conductive than air, it increases the thermal conductivity at the contact area between the heat sink 30 and the Peltier cell 52, allowing for the maximum heat transfer.

Preferably, the core technology units work as follows: the Peltier cell 52 works as the main method of cooling. When the system is turned on, the Peltier cell 52 transfers heat away from the body of the user to far surface of the plate 56a or 56b. At this point the entire Peltier cell 52 begins to heat up from the heat on the Peltier cell 52 hot side plate 56a or 56b. However, the heat sink 30 acts as a heat absorber, keeping the entire Peltier cell 52 from heating up. At some point, the heat sink 30 would begin to heat up except that the fan 40 passes air through the fins 36 of the heat sink 30, keeping the heat sink 30 cool. With such an arrangement of the core technology, the Peltier cell 52 cold side plate 56a or 56b reaches target temperatures, mimicking the cold sensation created by icing the body.

Heat Energy Transfer to the Body

Preferably, the Peltier cells 52 employed in the preferred embodiments have a relatively small surface area. Desirably, the surface area of each plate 56a, 56b of the cell 52 is approximately 2.25 in² to provide reasonable power requirements and for portability of the entire device 20. However, as it is often desirable to heat and cool areas larger than 2.25 in², it is beneficial to employ an intermediary, such as the fluid reservoir 28, that could transmit the heat, to or from the body, via the Peltier cell 52. It is preferable that the fluid, preferably a liquid, maintains its physical properties at both ends of the target temperature spectrum of about 46° C. and about 0° C.

Water, isopropyl alcohol and vegetable oil were tested within a seal plastic pouch. These pouches varied in size from 8 in² to 15 in² with liquid volumes of about 20 ml to about 100 ml. A series of heating and cooling tests were run to learn if any of the substances demonstrated an inability to transfer heat quickly and completely. It was quickly determined that water was not an ideal candidate as its high specific heat, 4.184 KJ/Kg*° C., meant that it required large amounts of heat pumped into it, or from it, to change the temperature significantly. The only way to produce the kind of heat transfer necessary to accomplish this is to run the system at with very high currents at high voltages, which is an undesirable situation due to the power usage and heat build up within the Peltier cell 52.

However, the relative low specific heats of both alcohol, 2.4 KJ/Kg*° C.7, and vegetable oil, 3.6 KJ/Kg*° C., made them each a viable alternative. Both of these substances were tested using a pouch size of about 2.5×5 inches with about 40 ml of liquid. After testing, an increase in the viscosity of the vegetable oil was observed at lower temperatures. The congealing of the vegetable oil made it less appealing than alcohol, which maintained its low viscosity for all temperatures. As a result, preferably, the preferred fluid reservoirs 28 employ a fluid 34 including alcohol. One preferred fluid 34 is a solution made of about 91% Isopropyl Alcohol and about 9% water, which is widely and cheaply available. When the system was running, it was discovered that the alcohol reached both the high and low temperatures in very little time. In addition, it was discovered that it also quickly returned to room temperature when the system was shut off, allowing for quick turnaround from the hot setting to the cold setting, likely due in large part to its low specific heat. Thus, although an alcohol based fluid is preferred, other suitable fluids may be used as well.

Vest for Back Therapy

FIG. 5 illustrates a preferred therapy device, which employs multiple therapy units 24, each including a fan 40, secured to a support 22 in the form of a vest 70. The vest 70 preferably is designed to fit on the back of a human male. Preferably, the vest 70 extends over the lower portion of the back to cover target muscle groups in the lower back, such as the Lattissimus Dorsi muscles, for example. The vest 70 is but one possible configuration of the support 70, as noted in the discussion above with reference to FIG. 1.

The vest 70 includes two side pockets 72 configured to hold two side pouches 32 (FIG. 1). Preferably, each of the side pouches is about 2.5×4.5 inches and filled with about 35 ml of alcohol solution, as described above. A rear pocket 74 extends across the lower back and holds a rear pouch 32 (FIG. 1). The rear pouch 32 preferably is about 2.5×8 inches and contains about 60 ml of alcohol solution.

The therapy units 24 preferably are stitched into the outer lining of the vest 70. Advantageously, by sewing the fabric tightly around the body of each unit 24, the units 24 are held firmly in place despite all of the movement involved in putting on, and taking off, the vest 70. Inside of the vest 70, each Peltier cell 56 (FIG. 2) is in direct contact with the pouch 32 via thermal compound layer 40. Each of the therapy units 24 preferably is connected through simple circuitry, with the main wire for the circuits preferably extending out of the vest 70 at a convenient location, such as the left shoulder of the vest 70, for example.

Electronics and Circuitry

Preferably, the circuitry for the vest 70 includes two small, independent circuits: one circuit 80 for the fans 40 and one circuit 82 for the thermoelectric devices 26 (or Peltier cells 52), as illustrated in FIG. 6. Preferably, each of the fans 40 is connected in parallel. Together, they require a 12V power source and 1.5 A of total current, roughly 0.5 A each. The circuit 82 for the Peltier cells 52 preferably is a series circuit with two different settings: hot and cold. On the cold setting, 2.6 A are required to reach target temperature and the circuit draws 36V. For the hot setting, the target temperature requires 0.6 A and the circuit draws the same 36V. The voltage of the hot setting has been denoted as negative.

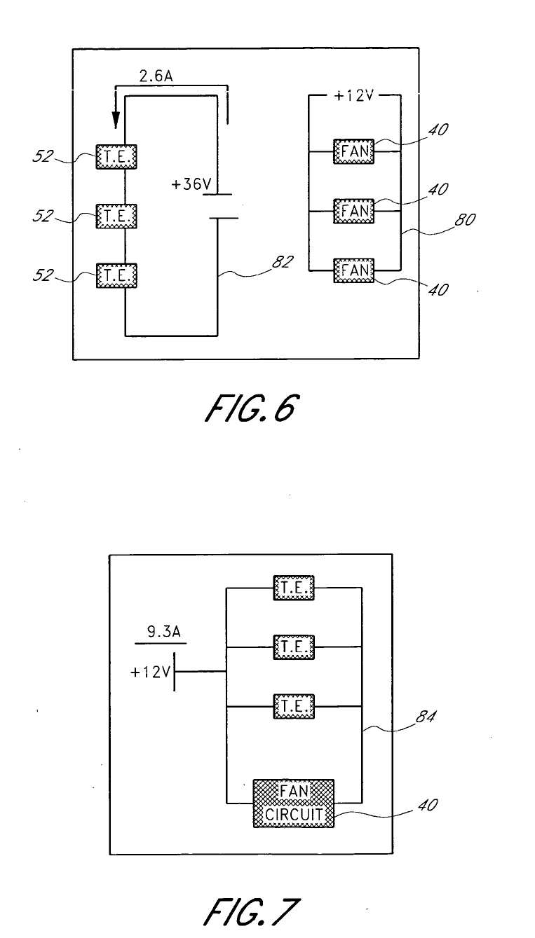

The above-described circuit 82 requires roughly a 36V-power supply, as there is about a 12V drop across each of the cells 52. However, if the wiring were redone as a parallel circuit 84, as illustrated in FIG. 7, the entire system would require only a 12V power supply. This is the voltage of the average car cigarette adapter. Such a circuit 84 would require a larger 9.3 A of current; however, this is within the capability of a cigarette lighter and car battery.

Therapy Unit Capabilities

A series of tests were completed to determine the temperature limits of the therapy device 20. The target temperatures were set from the beginning at 46° C. and 0° C. However, in some circumstances, it may be desirable for the therapy unit 24 to reach beyond these targets to see if the unit 24 was capable of a larger temperature range. Upon testing the therapy device 20, it was found that running 3.6 A on the hot setting, the therapy unit 24 produced a temperature of 187° C. within 10 seconds. On the cold setting, a current of 3.6 A produced a temperature of −19° C. almost immediately after being switched on.

Another advantageous feature about this therapy device 20 is that it quickly returns to room temperature. Running 3.6 A on the hot setting for 10 minutes, the therapy unit 24 again reached and maintained a temperature of 187° C. After turning the switch off, the Peltier cell 52 surface had returned to room temperature (26° C.) within 2 full minutes. Switching the therapy device 20 to cold and running it at 3.6 A, the therapy unit 24 reached its peak temperature of −19° C. After the running the therapy device 20 for 20 minutes, upon shutting it off, it was found that the surface of the Peltier cell 52 returned to room temperature in less than 1 minute.

Human Trials

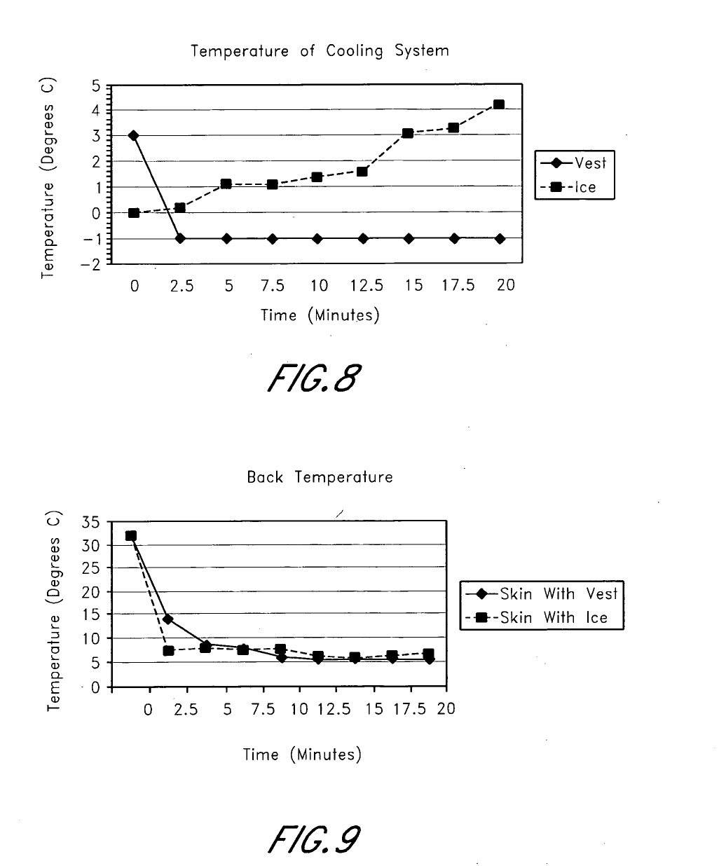

Two separate trials of each of the traditional cooling method of an ice pack and the therapy vest 70 were conducted. The procedure was simple: place the cooling system on the back and check skin and pack temperature in 2.5-minute intervals for twenty minutes. This procedure was carried out twice for a bag of ice and twice for the therapy vest 70. FIG. 8 is a graph of the Temperature of the Cooling systems vs. Time and FIG. 9 is a graph of the Skin Temperature vs. time.

In FIG. 8, it can be seen that the therapy vest 20 provides a steady performance with respect to cooling system temperature. Although the vest 70 starts at a higher temperature than the bag of ice, after 2.5 minutes, the vest 70 reaches a temperature of −1° C. The vest 70, and specifically a therapy unit 24 of the vest, remained at that temperature for the next seventeen and a half minutes of the trial. Contrasting this, after the same initial 2.5 minute time period the vest 70 required to cool down, the bag of ice had already begun to heat up. The increase of temperature in the bag of ice caused the ice to melt, leaving a bag with 2 in of water by the end of the 20-minute trial.

Referencing FIG. 9, it is apparent that the vest 70 performs as well as the bag of ice. Although there is a slight 3.5-minute delay, the back treated by the vest 70 reaches the temperature range achieved by the bag of ice. Towards the end of the 20-minute trial, the ice in the bag began to melt and the graph shows a slight warming of the skin on the back. The vest 70, however, maintained its temperature of −1° C. and maintained the cold back temperature through the entire trial time and beyond. Thus, the vest 70 was successful in mimicking ice in both temperatures achieved and sensations provided to the target area.

Advantageously, the primary components of the therapy device 20, such as the Peltier cells 52, heat sinks 30 and fans 40 are commonly available, lending to the affordability of the device 20 in all of its possible forms, including the vest 70. In terms of convenience, because the vest 70 is self-contained, it can be transported and worn almost anywhere. At most, while cooling, the preferred embodiment of the vest 70 only uses 100 W of energy, which is what is needed to light a light bulb. This low energy consumption makes adapting the system for travel possible.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In particular, while the present therapy device has been described in the context of particularly preferred embodiments, such as the vest, the skilled artisan will appreciate, in view of the present disclosure, that certain advantages, features and aspects of the device may be realized in a variety of other applications, many of which have been noted above. Additionally, it is contemplated that various aspects and features of the invention described can be practiced separately, combined together, or substituted for one another, and that a variety of combination and subcombinations of the features and aspects can be made and still fall within the scope of the invention. For example, it is contemplated that the core technology of the present therapy unit could be adapted for other uses, such as a portable icebox or cooler to keep food and/or beverages hot or cold, a blanket or variable temperature vehicle seats, for example. Other applications will be apparent to those of skill in the art in view of the present disclosure. Thus, it is intended that the scope of the present invention herein disclosed should not be limited by the particular disclosed embodiments described above, but should be determined only by a fair reading of the claims.

What is claimed is:

1. A variable temperature therapy system for permitting ambulatory use on an animal, comprising:
a support adapted to surround a portion of the body of a user of the device, the support securable to the body of the user;
a plurality of therapy units coupled to the support, the therapy units located on the support so as to be positioned proximate target treatment areas of the body of the user when the device is secured to the user, each temperature therapy unit comprising:
a resilient pouch containing a non-circulating volume of a liquid, the resilient pouch secured relative to the support;
a thermoelectric heat pump in thermal communication with the resilient pouch;
a heat sink coupled to a surface of the thermoelectric heat pump;
a fan configured to circulate air over the heat sink;
wherein the plurality of thermoelectric heat pumps are electrically connected with one another by an electric circuit, the electric circuit connectable to a power supply.

2. The therapy device of claim 1, wherein the support is a vest and wherein the plurality of therapy units comprises at least three therapy units, a first of the therapy units positioned in a center of a lower back portion of the vest, and a second and a third of the therapy units positioned on opposing sides of the first therapy unit such that the device facilitates therapy of a user's lower back.

3. The therapy device of claim 1, wherein the plurality of thermoelectric heat pumps are connected in series.

4. The therapy device of claim 1, wherein the plurality of thermoelectric heat pumps are connected in parallel.

5. The therapy device of claim 1, wherein the resilient pouch, the thermoelectric heat pump and at least a portion of the heat sink of each therapy unit are positioned within a space between a first layer and a second layer of the support.

6. The therapy device of claim 5, wherein an entirety of the fan of each therapy unit is external of the space.

7. The therapy device of claim 1, wherein a volume of the liquid within the resilient pouch of each therapy unit is between about 20 milliliters to 100 milliliters.

8. A self-contained variable temperature therapy device for use on an animal, comprising:
a support adapted to surround a portion of the body of a user of the device, the support securable to the body of the user;
a resilient pouch containing a volume of a liquid, the resilient pouch secured relative to the support so as to be proximate the body of the user when the support is secured to the user;
a thermoelectric heat pump, the thermoelectric heat pump connectable to a source of power;
a heat sink having a first thermal resistance;
a thermal compound layer interposed between the heat sink and a surface of the thermoelectric heat pump, the thermal compound having a second thermal resistance that is less than or equal to the first thermal resistance; and
a cooling device configured to withdraw heat from the heat sink.

9. The therapy device of claim 8, further comprising a second thermal compound layer interposed between a second surface of the thermoelectric heat pump and the flexible pouch, the second thermal compound layer having a third thermal resistance that is less than or equal to the first thermal resistance.

10. The therapy device of claim 8, wherein the thermal compound comprises a material containing silver.

11. The therapy device of claim 8, wherein the flexible pouch, the thermoelectric heat pump and at least a portion of the heat sink are positioned within a space between a first layer and a second layer of the support.

12. The therapy device of claim 8, wherein a volume of the liquid within the resilient pouch is between about 20 milliliters to 100 milliliters.

13. The therapy device of claim 8, wherein the support is a vest.

14. A variable temperature therapy device, comprising:
a support adapted to surround a portion of the body of a user of the device, the support securable to the body of the user and having a therapy surface configured to be adjacent a skin surface of the user when the device is in use;
a resilient pouch containing a non-circulating volume of a liquid, the resilient pouch secured relative to the support;
a thermoelectric heat pump in thermal communication with the resilient pouch on an opposite side of the resilient pouch from the body of the user when the device is in use, the thermoelectric heat pump connectable to a source of power;
a heat sink coupled to a surface of the thermoelectric heat pump;
a fan configured to circulate air over the heat sink;
wherein the device is configured such that the therapy surface is capable of maintaining a temperature at or below about 0 degrees Celsius for at least 20 minutes in a first setting and is capable of maintaining a temperature at or above about 46 degrees Celsius for at least 20 minutes in a second setting.

15. The therapy device of claim 14, further comprising a thermal compound layer interposed between the surface of the thermoelectric heat pump and the heat sink, wherein the thermal resistance of the thermal compound layer is no greater than the thermal resistance of the heat sink.

16. The therapy device of claim 14, wherein the fan and the heat sink are configured for an air flow rate over the heat sink of at least about 102 cubic feet per minute.

17. The therapy device of claim 14, wherein the liquid in the resilient pouch is an alcohol solution.

18. The therapy device of claim 14, wherein a volume of the liquid within the resilient pouch of each therapy unit is between about 20 milliliters and 100 milliliters.

19. The therapy device of claim 18, wherein the volume of the liquid is between about 35 milliliters and 60 milliliters.
